SEQUENCE LISTING (9 sequences)

SEQ ID NO: 1 - DNA, 20 bp, synthetic construct
  source 1..20: mol_type = other DNA
cctgtagcccacgtcgtagc

SEQ ID NO: 2 - DNA, 20 bp, synthetic construct
  source 1..20: mol_type = other DNA
ttgacctcagcgctgagttg

SEQ ID NO: 3 - DNA, 21 bp, synthetic construct
  source 1..21: mol_type = other DNA
atgcctgaagaccctgccaag

SEQ ID NO: 4 - DNA, 21 bp, synthetic construct
  source 1..21: mol_type = other DNA
ggtcagttagccttgcctttg

SEQ ID NO: 5 - DNA, 18 bp, synthetic construct
  source 1..18: mol_type = other DNA
ccgaagtcatagccacac

SEQ ID NO: 6 - DNA, 18 bp, synthetic construct
  source 1..18: mol_type = other DNA
gtgccatcagagcagtct

SEQ ID NO: 7 - DNA, 24 bp, synthetic construct
  source 1..24: mol_type = other DNA
agaatatctttcaggacccctgct

SEQ ID NO: 8 - DNA, 23 bp, synthetic construct
  source 1..23: mol_type = other DNA
acaccagtgctgagcatactttt

SEQ ID NO: 9 - DNA, 26 bp, synthetic construct
  source 1..26: mol_type = other DNA
catgttccagtatgactccactcacg

(19) United States
(12) Patent Application Publication
(10) Pub. No.: US 2025/0049873 A1
(43) Pub. Date: Feb. 13, 2025

(54) COMPOSITION FOR PREVENTING, AMELIORATING OR TREATING RESPIRATORY DISEASE COMPRISING LYSIMACHIA MAURITIANA EXTRACT AS EFFECTIVE COMPONENT

(71) Applicant: KOREA INSTITUTE OF ORIENTAL MEDICINE, Daejeon (KR)

(72) Inventors: YOON-YOUNG SUNG, Daejeon (KR); DONG-SEON KIM, Daejeon (KR); EUN JUNG SON, Sejong-si (KR); HEUNG JOO YUK, Daejeon (KR); YUN MI LEE, Daejeon (KR); YOUNG SOOK KIM, Daejeon (KR)

(21) Appl. No.: 18/722,979

(22) PCT Filed: Dec. 22, 2022
(86) PCT No.: PCT/KR2022/021043

§ 371 Date: Jun. 21, 2024

(30) Foreign Application Priority Data
Dec. 23, 2021 (KR) 10-2021-0186397

(51) Int. Cl. A61K 36/185; A61P 11/00

(52) U.S. Cl. CPC A61K 36/185; A61P 11/00; A61K 2236/331

(57) ABSTRACT

In a method for ameliorating or treating a respiratory disease, a composition including a Lysimachia mauritiana extract as effective component is administered to a subject in need thereof. Since the Lysimachia mauritiana extract is effective in reducing the production amount of the chemokine RANTES, an inflammatory mediator, from bronchial epithelial cells, which are respiratory cells, the composition can be advantageously used as a functional health food or medicine for respiratory diseases including asthma or chronic obstructive pulmonary disease (COPD).

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/021043, filed Dec. 22, 2022, which claims priority to the benefit of Korean Patent Application No. 10-2021-0186397 filed in the Korean Intellectual Property Office on Dec. 23, 2021, the entire contents of which are incorporated herein by reference.

### REFERENCE TO AN ELECTRONIC SEQUENCE LISTING

A sequence listing electronically submitted on Jun. 21, 2024, as a XML file named 20240621_535224GR04_TU_SEQ.XML, created on May 17, 2024 and having a size of 9,111 bytes, is incorporated herein by reference in its entirety.

### BACKGROUND

### 1. Technical Field

The present invention relates to a composition for preventing, ameliorating or treating a respiratory disease comprising Lysimachia mauritiana extract as effective component.

### 2. Background Art

Asthma, chronic obstructive pulmonary disease, allergic rhinitis, phlegm cough, acute and chronic bronchitis, bronchiolitis, laryngitis, pharyngitis, and tonsillitis are the representative examples of a respiratory disease. Asthma specifically refers to chronic inflammation occurring in the airways, especially in the bronchi. Inflammation triggered by asthma can be aggravated by various factors such as smoke, allergic antigens, cold air, physical exercise, respiratory infections, and more. Persistent inflammation leads to structural changes and hyper-responsiveness of the airways. As caused by these factors, symptoms such as wheezing (i.e., symptom showing a whistling or rattling breath sound caused by narrowed airways), shortness of breath, coughing, and excessive phlegm production are commonly exhibited.

The respiratory airway consists primarily of mucous membranes and muscles called bronchial smooth muscles. The mucous membranes contain numerous glands that continuously secrete necessary fluids. When the smooth muscles of the bronchi contract, the airway is narrowed. Once the inflammation is caused by various factors such as smoke, allergic antigens, cold air, physical exercise, and respiratory infections, an increased secretion from the glands is triggered, causing the fluid to block the airway and swelling of the mucous membranes toward the inside of the airway, consequently narrowing it further. As a result, sudden attacks of coughing accompanied by wheezing and severe difficulty in breathing occur. During an attack, dry coughing may occur and a sensation of chest tightness is often felt. There are also many cases of asthma caused by unknown factors showing just a difficulty in breathing with chronic coughing and chest tightness without wheezing, and these symptoms tend to occur suddenly in everyday life.

Meanwhile, Lysimachia mauritiana is a dicotyledonous plant found in coastal rocky areas in the southern region of South Korea, including Jeju Island and Ulleung Island. Young leaves of Lysimachia mauritiana are edible and the fruit is used as an ornamental. In oriental medicine, it is used as a tonic and diuretic, and is known to be effective in treating high blood pressure, diabetes, constipation, edema, and bruising.

Regarding the techniques related to Lysimachia mauritiana extract, Korean Patent Registration No. 1168567 describes the anti-fungal activity of saponin-based substances that are contained in Lysimachia mauritiana. However, as of now, there has been no disclosure of a composition for preventing, ameliorating or treating a respiratory disease comprising Lysimachia mauritiana extract as effective component as described in the present invention.

### SUMMARY

The present invention is devised under the aforementioned circumstances, and provided by the present invention is a composition for preventing, ameliorating or treating a respiratory disease comprising Lysimachia mauritiana extract as effective component. It was found in the present invention that the extract of Lysimachia mauritiana is effective not only in reducing the production amount of chemokine RANTES, which is an inflammatory mediator, from bronchial epithelial cells as respiratory cells and reducing the expression level of inflammatory cytokines (IL-6, TNF-α, and MUC5AC) but also in reducing the total number of BAL (bronchoalveolar lavage) cells and the number of neutrophils in BAL, and reducing the content of IL-1α, TNF-α, IL-17, MIP2, and CXCL-1 in BALF (bronchoalveolar lavage fluid) and gene expression level of TNF-α, MIP2, CXCL-1, and MUC5AC, and it is further found that the extract of Lysimachia mauritiana exhibits the effect of suppressing lung tissue damage. Based on those findings, the present invention is completed accordingly.

To achieve the object described in the above, the present invention provides a functional health food composition for preventing or ameliorating a respiratory disease comprising Lysimachia mauritiana extract as effective component.

The present invention further provides a pharmaceutical composition for preventing or treating a respiratory disease comprising Lysimachia mauritiana extract as effective component.

The present invention further provides a veterinary composition for preventing or treating a respiratory disease comprising Lysimachia mauritiana extract as effective component.

The present invention still further provides an animal feed additive for preventing or ameliorating a respiratory disease comprising Lysimachia mauritiana extract as effective component.

The present invention relates to a composition for preventing, ameliorating or treating a respiratory disease comprising Lysimachia mauritiana extract as effective component. The extract of Lysimachia mauritiana is found to be effective not only in reducing the production amount of chemokine RANTES, which is an inflammatory mediator, from bronchial epithelial cells as respiratory cells and reducing the expression level of inflammatory cytokines (IL-6, TNF-α, and MUC5AC) but also in reducing the total number of BAL cells and the number of neutrophils in BAL, and reducing the content of IL-1α, TNF-α, IL-17, MIP2, and CXCL-1 in BALF and gene expression level of TNF-α, MIP2, CXCL-1, and MUC5AC, and the extract of Lysimachia mauritiana is further found to exhibit the effect of suppressing lung tissue damage.

### BRIEF DESCRIPTION OF THE DRAWINGS

FIG. 1 shows the results of determining the production rate of RANTES in bronchial epithelial cells (BEAS-2B) after a treatment with the extract of Lysimachia mauritiana. Dexa is a positive control group treated with 0.5 μM of dexamethasone. ###indicates a statistically significant increase (p<0.001) in the RANTES production rate in the 10 ng/ml TNFα treatment group (TNFα) compared to the control group (Control), and *** indicates a statistically significant decrease (p<0.001) in the RANTES production rate in the group treated with the extract of Lysimachia mauritiana and the positive control group (Dexa) compared to the group treated with 10 ng/ml TNF-α.

FIG. 2 shows the results of determining changes in the expression level of the inflammatory cytokine IL-6 after inducing inflammation with LPS in bronchial epithelial cells. ###indicates a statistically significant increase (p<0.001) in IL-6 expression level in the LPS treatment group (LPS20) compared to Control (normal control group), while*and *** indicate a statistically significant decrease in the expression level of IL-6 in the group treated with 25 or 50 μg/ml extract of Lysimachia mauritiana of the present invention (LM25, LM50) compared to the LPS group, in which*has p<0.05, and *** has p<0.001.

FIG. 3 shows the results of determining changes in the expression level of the inflammatory cytokine TNF-α after inducing inflammation with LPS in bronchial epithelial cells. ###indicates a statistically significant increase (p<0.001) in TNF-α expression level in the LPS treatment group (LPS20) compared to Control (normal control group), while*** indicates a statistically significant decrease (p<0.001) in the expression level of TNF-α in the group treated with 50 μg/ml extract of Lysimachia mauritiana of the present invention (LM50) compared to the LPS group.

FIG. 4 shows the results of determining changes in the expression level of the inflammatory cytokine MUC5AC after inducing inflammation with LPS in bronchial epithelial cells. ###indicates a statistically significant increase (p<0.001) in MUC5AC expression level in the LPS treatment group (LPS20) compared to Control (normal control group), while*** indicates a statistically significant decrease (p<0.001) in the expression level of MUC5AC in the group treated with 50 μg/ml extract of Lysimachia mauritiana of the present invention (LM50) compared to the LPS group.

FIG. 5 is a schematic diagram showing the experimental schedule for determining the effect of the extract of Lysimachia mauritiana of the present invention using an animal model of respiratory damage.

FIG. 6 shows the results of determining the total number of bronchoalveolar lavage (BAL) cells and the total number of lung cells after administering the extract of Lysimachia mauritiana of the present invention to an animal model of respiratory damage. ###indicates a statistically significant increase (p<0.001) in the total number of BAL cells or the total number of lung cells in the control group with respiratory damage induced by particulate matter (i.e., fine dust) compared to the normal group, while * and *** indicate a statistically significant decrease in the total number of BAL cells or the total number of lung cells in the group administered with the extract of Lysimachia mauritiana of the present inventio or the positive control group compared to the control group, in which*has p<0.05, and *** has p<0.001.

FIG. 7 shows the results of determining the number of neutrophils in BAL after administering the extract of Lysimachia mauritiana of the present invention to an animal model of respiratory damage. ###indicates a statistically significant increase (p<0.001) in the number of neutrophils in BAL in the control group with respiratory damage induced by particulate matter (i.e., fine dust) compared to the normal group, while *, **, and *** indicate a statistically significant decrease in the number of neutrophils in BAL in the group administered with the extract of Lysimachia mauritiana of the present invention or the positive control group compared to the control group, in which*has p<0.05, ** has p<0.01, and *** has p<0.001.

FIGS. 8A to 8D show the results of determining (FIG. 8A) the number of CD4⁺ and CD8⁺ in BAL, (FIG. 8B) the number of Gr⁻1⁺SiglecF⁻ neutrophils in BAL, (FIG. 8C) Gr⁻1⁺CD11b⁺ granulocytes in lung, and (FIG. 8D) the number of Gr⁻1⁺SiglecF⁻ neutrophils in lung, all after administering the extract of Lysimachia mauritiana of the present invention to an animal model of respiratory damage. ##and ###indicate a statistically significant increase in the number of immune cells in BAL or lung of the control group with respiratory damage induced by particulate matter (i.e., fine dust) compared to the normal group, in which ##has p<0.01 and ###has p<0.001. *, **, and *** indicate a statistically significant decrease in the number of immune cells in the group administered with the extract of Lysimachia mauritiana of the present invention or the positive control group compared to the control group, in which*has p<0.05, ** has p<0.01, and *** has p<0.001.

FIG. 9 shows the results of determining the content of IL-la, TNF-α, IL-17, MIP2, and CXCL-1 in BALF after administering the extract of Lysimachia mauritiana of the present invention to an animal model of respiratory damage. ##and ###indicate a statistically significant increase in the content of IL-1α, TNF-α, IL-17, MIP2, and CXCL-1 in BALF in the control group with respiratory damage induced by particulate matter (i.e., fine dust) compared to the normal group, in which ##has p<0.01 and ###has p<0.001. *, **, and *** indicate a statistically significant decrease in the content of IL-1α, TNF-α, IL-17, MIP2, and CXCL-1 in BALF in the group administered with the extract of Lysimachia mauritiana of the present invention or the positive control group compared to the control group, in which*has p<0.05, ** has p<0.01, and *** has p<0.001.

FIG. 10 shows the results of determining the mRNA expression level of TNF-α, MIP2, CXCL-1, and MUC5AC in lung tissues after administering the extract of Lysimachia mauritiana of the present invention to an animal model of respiratory damage. ##and ###indicate a statistically significant increase in the gene expression level of TNF-α, MIP2, CXCL-1, and MUC5AC in lung tissues of the control group with respiratory damage induced by particulate matter (i.e., fine dust) compared to the normal group, in which ##has p<0.01 and ###has p<0.001. *, **, and *** indicate a statistically significant decrease in the gene expression level of TNF-α, MIP2, CXCL-1, and MUC5AC in lung tissues of the group administered with the extract of Lysimachia mauritiana of the present invention or the positive control group compared to the control group, in which*has p<0.05, ** has p<0.01, and *** has p<0.001.

FIG. 11 shows the results of H&E staining and M-T staining for determining the suppressed pathological damage to lung tissue as a result of administering the extract of Lysimachia mauritiana of the present invention to an animal model of respiratory damage.

### DETAILED DESCRIPTION

The present invention relates to a functional health food composition for preventing or ameliorating a respiratory disease comprising Lysimachia mauritiana extract as effective component.

The respiratory disease is preferably selected from the group consisting of asthma, chronic obstructive pulmonary disease, bronchitis, pharyngitis, tonsillitis, and laryngitis, but it is not limited to them.

The Lysimachia mauritiana extract is preferably obtained by extracting the aerial part of Lysimachia mauritiana, but is not limited thereto, and any specific part of the plant can be selected and used as needed.

The Lysimachia mauritiana extract can be produced by a method including the following steps:
(1) carrying out extraction by adding an extraction solvent to dry Lysimachia mauritiana;
(2) filtering the extract of the step (1); and
(3) drying the extract filtered in the step (2) to give extract, but the method is not limited thereto.

The extraction solvent of the above step (1) is preferably selected from water, C₁-C₄ lower alcohol, and a mixture thereof. It is more preferably water or ethanol, and even more preferably ethanol, but it is not limited thereto. With regard to the production method described in the present invention, any kind of common methods that are generally known as extraction method in the pertinent art, e.g., hot water extraction, impregnation extraction, extraction by reflux condensation, and ultrasonic extraction, can be used. It is preferable that the extraction is carried out by adding the extraction solvent in an amount of 1 to 20 times the weight of Lysimachia mauritiana. The extraction temperature is preferably between 20° C. and 100° C., but it is not limited thereto. Furthermore, the extraction time is preferably between 1 hour and 5 hours and more preferably between 1 hour and 3 hours, but it is not limited thereto. It is preferable that the drying of the step (3) above is carried out by drying under reduced pressure, drying under vacuum, drying under boiling, spray drying, or freeze-drying, and it is more preferably carried out by freeze-drying, but it is not limited thereto.

The functional health food composition is preferably prepared in any one formulation selected from powder, granule, pill, tablet, capsule, candy, syrup, and beverage, but it is not limited thereto. The functional health food composition of the present invention can be prepared by adding the Lysimachia mauritiana extract by itself or mixing it with other food product or other food component. The composition can be suitably prepared by following a common method. Examples of the food product to which the Lysimachia mauritiana extract of the present invention can be added include caramel, meat, sausage, bread, chocolate, candies, snacks, biscuits, pizza, ramen, other noodles, gums, dairy products including ice cream, various kinds of soup, beverage, tea, drink, alcohol beverage, and vitamin complex, and all functional health food products in general sense are included therein. In other words, the type of the food products is not particularly limited. The functional health food composition may further comprise various nutritional supplements, a vitamin, a mineral (i.e., electrolyte), a synthetic or natural flavor, a coloring agent, an enhancer (i.e., cheese, chocolate, etc.), pectinic acid and a salt thereof, alginic acid and a salt thereof, an organic acid, a protective colloidal thickening agent, a pH adjusting agent, a stabilizer, a preservative, glycerin, alcohol, and a carbonating agent used for carbonated beverage. Other than those, fruit pulp for producing fruit juice or vegetable beverage may be additionally comprised. Those components may be used either independently or in combination thereof.

The functional health food composition of the present invention may further include various flavorings or natural carbohydrates as additional ingredients. The natural carbohydrates may include monosaccharides such as glucose and fructose, disaccharides such as maltose and sucrose, and polysaccharides such as dextrin and cyclodextrin, as well as sugar alcohols such as xylitol, sorbitol, and erythritol. The ratio of the natural carbohydrates is, although not critically important, preferably about 0.01 g to 0.04 g, more preferably about 0.02 g to 0.03 g per 100 g of the composition of the present invention, but it is not limited thereto. As sweeteners, natural sweeteners such as thaumatin and Stevia extracts, or synthetic sweeteners such as saccharin and aspartame, can be used.

The present invention further relates to a pharmaceutical composition for preventing or treating a respiratory disease comprising Lysimachia mauritiana extract as effective component.

The pharmaceutical composition comprising the Lysimachia mauritiana extract according to the present invention preferably prepared in any one formulation selected from capsule, powder, granule, tablet, suspension, emulsion, solution, syrup, and aerosol, but it is not limited thereto. The pharmaceutical composition of the present invention may further comprise, in addition to the Lysimachia mauritiana extract, a pharmaceutically acceptable carrier, excipient, or diluent, and examples thereof include lactose, dextrose, sucrose, oligosaccharides, sorbitol, mannitol, xylitol, erythritol, maltitol, starch, gum acacia, alginate, gelatin, calcium phosphate, calcium silicate, cellulose, methyl cellulose, non-crystalline cellulose, polyvinylpyrrolidone, water, methylhydroxybenzoate, propylhydroxybenzoate, talc, magnesium stearate and mineral oil. When formulated, it can be prepared using diluents or excipients such as commonly used fillers, extenders, binders, wetting agents, disintegrants, and surfactants. The preferred dosage of the Lysimachia mauritiana extract of the present invention varies depending on the patient's condition and weight, severity of disease, drug form, administration route and period, but can be appropriately selected by a person skilled in the pertinent art. However, for a desirable effect, it is recommended that the pharmaceutical composition comprising the Lysimachia mauritiana extract of the present invention be administered at 0.0001 to 100 mg/kg per day, preferably at 0.001 to 10 mg/kg per day. Administration may be made once a day, or may be administered several times. The above-described dosage does not limit the scope of the present invention in any sense.

The present invention further relates to a veterinary composition for preventing or treating a respiratory disease comprising Lysimachia mauritiana extract as effective component.

The veterinary composition of the present invention may further comprise appropriate excipients and diluents according to conventional methods. Examples of the excipients and diluents that can be comprised in the veterinary composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, polyvinylpyrrolidone, water, methylhydroxybenzoate, propylhydroxybenzoate, talc, magnesium stearate, cetanol, liquid paraffin, sorbitan monostearate, polysorbate 60, methylparaben, propylparaben, and mineral oil. The veterinary composition according to the present invention may additionally include fillers, anticoagulants, lubricants, moistening agents, flavorings, emulsifiers, preservatives, and the like. The veterinary composition according to the present invention can be formulated using well-known methods in the industry to provide rapid, sustained, or delayed release of the active ingredient after administration to animals. The formulation can take various forms such as powder, granule, tablet, capsule, suspension, emulsion, solution, syrup, aerosol, soft or hard gelatin capsule, suppository, sterile injectable solution, or sterile topical preparation.

The effective amount of the veterinary composition according to the present invention can be suitably selected according to the individual characteristics of the animal. Specifically, the effective amount can be determined based on factors such as the severity of the disease or condition, the age, weight, health status, or gender of the animal, sensitivity to the effective component of the present invention, the route of administration, the duration of administration, other composition used in combination or simultaneously with the composition of the present invention, and other factors that are well known in the fields of physiology and veterinary medicine.

The present invention still further relates to an animal feed additive for preventing or ameliorating a respiratory disease comprising Lysimachia mauritiana extract as effective component.

The animal feed additive of the present invention corresponds to a supplementary animal feed in terms of the animal feed management regulations. In the present invention, the term‘animal feed’ may refer to any natural or artificial prescribed feed, single feed, or the components of such single feed, which animals eat, ingest, and digest. The types of the animal feed are not particularly limited and can include the feeds that are commonly used in the relevant technical field. Non-limiting examples of such feeds include plant-based feeds such as grains, root vegetables, by-products of food processing, algae, fibers, pharmaceutical by-products, oils, starches, gourd proteins, and cereal by-products; and animal-based feeds such as proteins, minerals, oils, mineral substances, fats, single-cell proteins, animal plankton, or food. These can be used either alone or in a mixture of two or more of them.

Hereinbelow, the present invention is explained in greater detail in view of Examples. However, the following Examples are given only for more specific explanation of the present invention and it is evident to a person who has common knowledge in the pertinent art that the scope of the present invention is not limited by them.

### EXAMPLES

Example 1. Preparation of Lysimachia mauritiana Extract

Aerial part of dry Lysimachia mauritiana was extracted, twice for 2 hours, by reflux using 50% (v/v) ethanol. Then, the extract was filtered and the filtered solution was concentrated and dried under reduced pressure to give the extract.

### Example 2. Determination of Inhibitory Activity on Respiratory Inflammation in Bronchial Epithelial Cells (BEAS-2B)

The inhibitory activity of Lysimachia mauritiana extract on respiratory inflammation was evaluated, by using human bronchial epithelial cell line, based on the activity of inhibiting the secretion of RANTES, which is a pro-inflammatory chemokine.

Human bronchial epithelial cell line BEAS-2B (ATCC, USA) cells were cultured with DMEM (Dulbecco's modified eagle's medium) added with fetal bovine serum (FBS) and penicillin-streptomycin (PS), and then the cells (5×10 cells/well) were incubated in DMEM medium containing 10% in a 96-well plate for 18 hours. After that, the medium was removed and replaced with serum-free DMEM, and the cells were treated simultaneously with 100 or 200 μg/ml of Lysimachia mauritiana extract and 10 ng/ml of TNF-α, and incubated for 24 hours. With regard to the secretion amount of RANTES, an ELISA kit (R&D systems, USA) was used according to the manufacturer's instructions to measure the amount of RANTE secreted in the cell supernatant. Statistical differences were assessed for significance by using the Student's t-test.

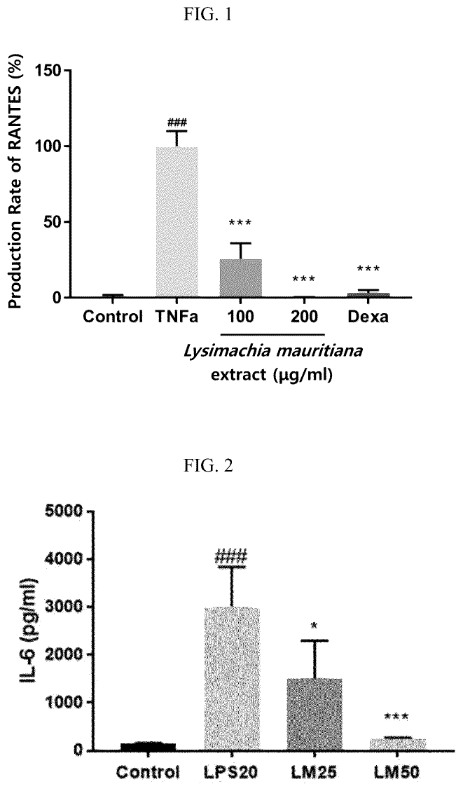

As a result, as shown in FIG. 1, the production amount of RANTES increased statistically significantly in the TNF-α treatment group compared to the control group (Control), while the production amount of RANTES was statistically significantly decreased in the group treated with the Lysimachia mauritiana extract of the present invention and also in the positive control group (Dexa).

Example 3. Determination of Changes in Expression Level of Inflammatory Cytokines IL-6 and TNF-α, and Mucus-Secreting Protein MUC5AC after Inducing Inflammation in Bronchial Epithelial Cells Using LPS

BEAS-2B cells, which are human bronchial epithelial cells (ATCC, USA), were incubated for 18 hours in DMEM medium added with FBS and penicillin-streptomycin (PS), then the medium was removed and replaced with serum-free DMEM. Thereafter, the cells were treated with a test sample (25 or 50 μg/ml) and LPS (20 μg/ml) for 24 hours. After the incubation for 24 hours, the cell supernatant was collected and the expression level of IL-6, TNF-α and MUC5AC was measured.

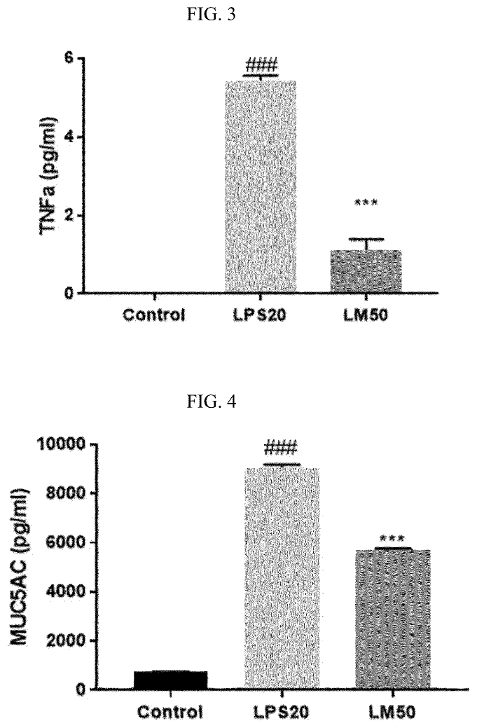

As a result, it was found that the expression level of IL-6, TNF-α and MUC5AC increased significantly in the LPS induction group compared to the control group (CON), but the expression level of IL-6, TNF-α and MUC5AC decreased significantly in the test group treated with Lysimachia mauritiana extract (FIGS. 2 to 4).

### Example 4. Determination of Effect of Ameliorating Respiratory Inflammation in Model of Respiratory Damage Induced by Fine Dust Complex

Using 7-week-old male BALB/c mice, an animal model of lung damage was prepared. Specifically, respiratory tract-damaging particulate matter complex [(PM10D); particulate matter (PM10-like, ERM-CZ120) and diesel exhaust particle (DEP)] was diluted in 1% alum and injected directly into the animal lung through the airway, three times (i.e., 3, 6, and 9 days after drug administration) at 3-day interval, using the Intro-Nazal-Tracheal (INT) injection method. The test group consisted of a normal group (Normal) that did not receive any treatment, a control group (CTL) that was induced to have respiratory damage by a treatment with particulate matter complex (PM10D), a positive control group that received particulate matter complex and dexamethasone (Dex_3 mg/kg), and Lysimachia mauritiana extract group (LM_50, LM_100) that received a particulate matter complex and Lysimachia mauritiana extract (50 mg/kg or 100 mg/kg).

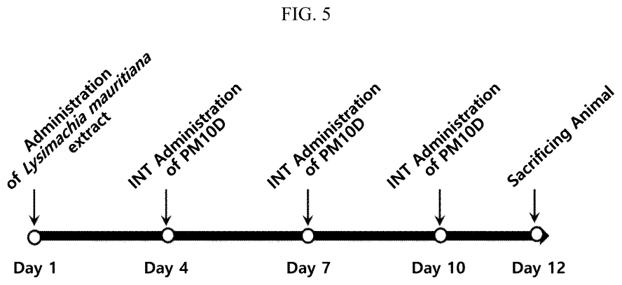

The test chemical was administered orally every day, and after completing the experiment, bronchoalveolar lavage fluid (BALF) and lung tissue were isolated from the mice of each group (FIG. 5).

### (1) Separation of Bronchoalveolar Lavage Fluid

On the last day of the experiment, after blood was drawn, a syringe containing 1 ml of FBS-free DMEM medium was inserted into the trachea by opening the chest and exposing the airway, and after fixing with a string, the bronchial alveolar lavage was carried out continuously 3 times to obtain a bronchoalveolar lavage solution (BALF). After centrifugation of the bronchoalveolar lavage fluid at 4° C., 2,000 rpm, for 5 minutes, the supernatant was preserved in frozen state, and the cells separated from the BALF were treated with ACK solution for 3 minutes to lyse red blood cells, then washed again with 1% FBS-free DMEM culture medium, and then the total cell count was measured using a hemocyte counter.

### (2) Separation of Bronchoalveolar Lavage Fluid (BALF) and Determination of Total Neutrophil Count

After drawing blood, the animal was dissected and centrifugation was carried out for counting neutrophils the BALF. Precipitated hemocytes were stained with 0.04% trypan blue and total cell count was measured. Diff-Quick staining (Romanowsky stain) was carried out three times, followed by two washes with PBS. Then, nine slides were produced for each group and counting was performed using an optical microscope (Light microscope, Nikon, Japan) at 400× magnification.

### (3) Separation of Lung Cells and Determination of Total Cell Count

Lung was harvested from a mouse from which the bronchoalveolar lavage fluid (BALF) had not been separated. The lung tissue was finely chopped, and then placed in 3 ml DMEM medium containing no fetal bovine serum (FBS), and, for separating the lung cells, 1 mg/ml collagenase IV (C5138, Sigma) was added to the lung cells for having, at least 4 times, tissue digestion in shaking incubator at 37° C. for 30 minutes or longer. The separated lung cells were washed with the medium and then passed through a cell filter to remove any undegraded tissues or impurities other than the desired cells. The resulting cells were treated with ACK solution (10-548E, LONZA) at 37° C. for 5 minutes to lyse red blood cells and washed again with the medium. Then, the cells were stained with 0.04% trypan blue (15250061, Invitrogen) and the total number of lung cells was measured. To investigate the inhibition of the increase in inflammation-related cells, the total cell count in BAL and the lung tissue was determined.

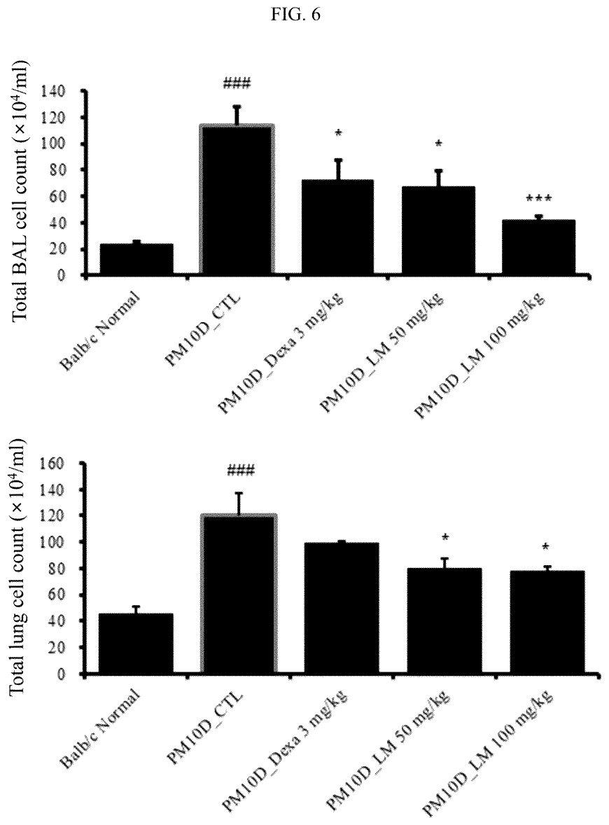

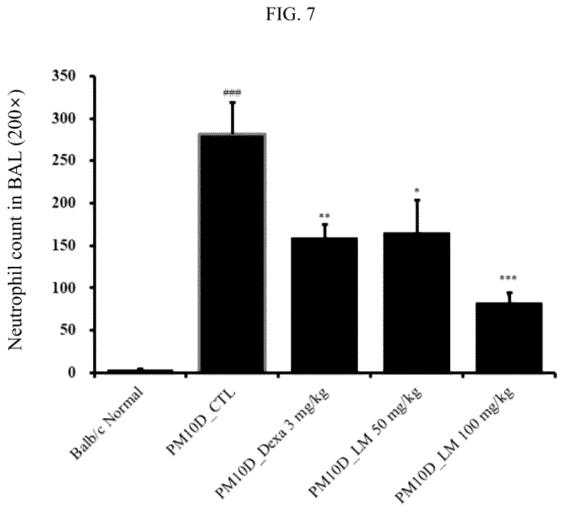

As a result, the total number of cells increased in BAL and the lung of the control group composed of mice with particulate matter-induced respiratory disease, and the number of neutrophils in BAL increased significantly, resulting in an increase in the level of inflammation in the lung. in contrast, the total number of cells in BAL, total number of lung cells, and the number of neutrophils in BAL decreased in the group treated with dexamethasone as positive control and also in the group administered with the Lysimachia mauritiana (LM) extract (FIGS. 6 and 7).

### (4) Immunofluorescence Cell Staining

The separated BAL and lung cells were subjected to immunofluorescence staining using CD4, CD8, Gr−1, SiglecF, and CDT 1b⁺ antibodies after adjusting the cell count, and the cell frequency was analyzed as a percentage (%) by using the FACS Calibur instrument (BD Biosciences, USA). Then, with an application of the total cell count, analysis was made for each tissue.

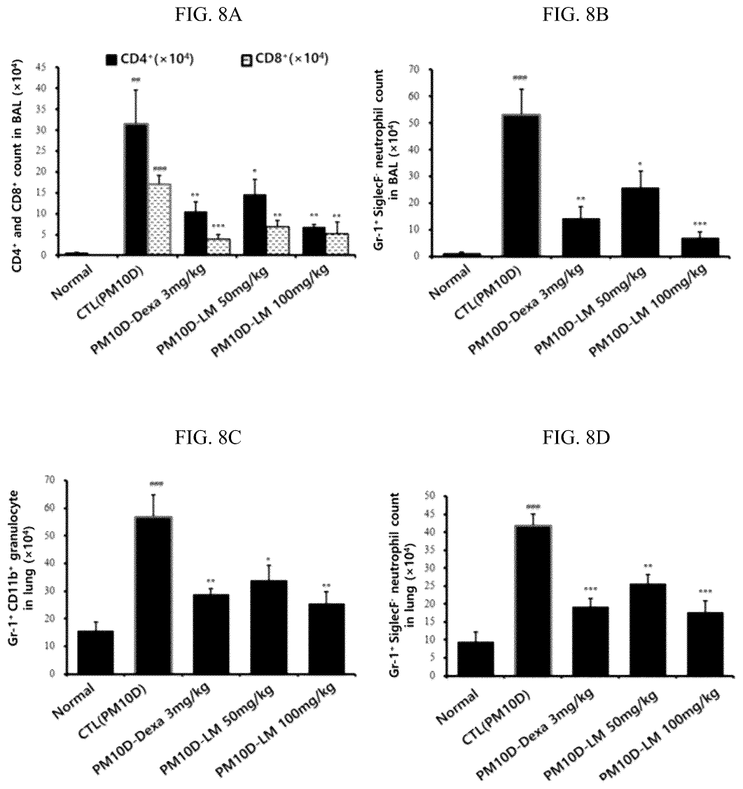

As a result of the cell frequency analysis and calculation based on cell sorting (FACS) in BAL, it was found that the number of CD4⁺CD8⁺ and Gr−1⁺SiglecF⁻ neutrophil immune cells increased in the control group, and the number of Gr⁻1⁺CD11b⁺ granulocyte and Gr⁻1⁺SiglecF⁻ neutrophil immune cells increased in the lung. In contrast, the number of immune cells decreased in the positive control group and the group administered with the Lysimachia mauritiana (LM) extract (FIGS. 8A to 8D).

### (5) Enzyme-Linked Immunosorbent Assay (ELISA)

ELISA was performed to measure the amount of IL-1α, TNF-α, IL-17, MIP2, and CXCL-1 in BALF. Capture antibodies were added, and after keeping the mixture overnight at 4° C., the resultant was washed 4 times with a washing buffer. Biotin-conjugated antibody reagent was added in an amount of 100 μl to each well, and the reaction was allowed to occur at room temperature for 1 hour. After washing twice, the resultant was treated with streptavidin-HRP solution in an amount of 100 μl in each well followed by a reaction at room temperature for 1 hour, and then washed again twice with washing buffer. After treating with 100 μl of substrate solution followed by a reaction for 20 minutes, 50 μl of reaction stop solution were added to terminate the reaction and the absorbance was measured at 450 nm.

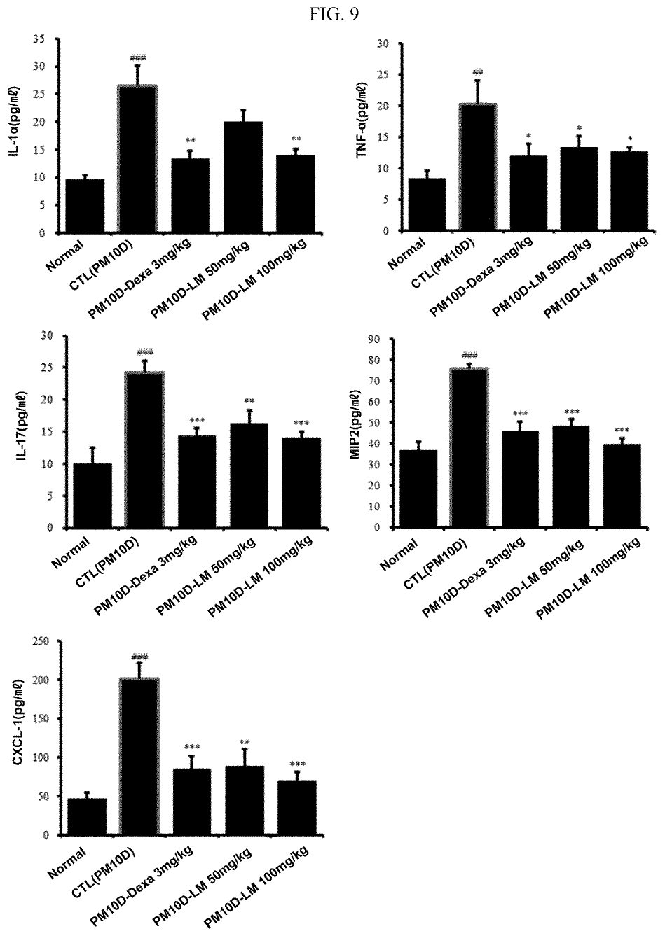

As a result, it was found that the production amount of the inflammation-related cytokine chemokines IL-1α, TNF-α, IL-17, MIP2, and CXCL-1 in BALF increased in the control group with the induction by the particulate matter, while it decreased statistically significantly in the positive control group and the group administered with the Lysimachia mauritiana (LM) extract (FIG. 9).

### (6) Determination of Gene Expression Level of TNF-α, MIP2, CXCL-1, and MUC5AC in Lung Tissue

After harvesting lung tissue from a mouse, 500 μl of RNA extraction solution were added and homogenization was carried out until the tissue is dissolved. After centrifugation at 13,000 rpm, RNA was extracted by washing and drying with ethanol. The extracted RNA was used for the synthesis of first strand cDNA.

The synthesized cDNA was subjected to a real-time polymerization chain reaction using Applied Biosystem 7500 Real-Time PCR System (Applied Biosystems, USA). Specifically, TNF-α, MIP2, CXCL-1, and MUC5AC primers were used, and G3PDH probe (Applied Biosystems, USA, VIC) was used. Real-time polymerization chain reaction conditions were as follows: 2 minutes at 50° C., 10 minutes at 94° C., and 40 cycles at 95° C. for 0.15 minutes and 60° C. for 1 minute. By using G3PDH as an internal standard, relative quantitative (RQ) was calculated for the group administered with the Lysimachia mauritiana extract and control group.

TABLE 1
Sequences of primers and probes

| | | | SEQ |
|---|---|---|---|
| Gene | Direction | Sequence (5′→3′) | ID NO: |
| TNF-α | Forward | CCTGTAGCCCACGTCGTAGC | 1 |
| | Reverse | TTGACCTCAGCGCTGAGTTG | 2 |
| MIP2 | Forward | ATGCCTGAAGACCCTGCCAAG | 3 |
| | Reverse | GGTCAGTTAGCCTTGCCTTTG | 4 |
| CXCL-1 | Forward | CCGAAGTCATAGCCACAC | 5 |
| | Reverse | GTGCCATCAGAGCAGTCT | 6 |
| MUC5AC | Forward | AGAATATCTTTCAGGACCCCTGCT | 7 |
| | Reverse | ACACCAGTGCTGAGCATACTTTT | 8 |
| G3PDH | Probe | CATGTTCCAGTATGACTCCACTCACG | 9 |

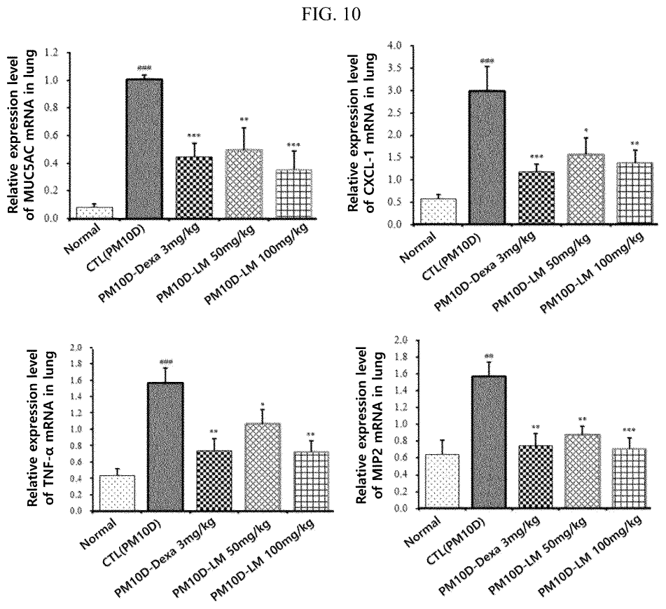

As a result, gene expression of the inflammation-related cytokines TNF-α, MIP2, and CXCL-1 and mucus protein MUC5AC showed an increase in the control group with induction by particulate matter, while it showed a significant decrease in the positive control group and the group administered with the Lysimachia mauritiana (LM) extract (FIG. 10).

### (7) Histopathological Examination

In order to observe the degree of pathological damage to lung tissue, lung tissue was removed from the test animal and fixed in 10% neutral-buffered formalin for 24 hours, then dehydrated with alcohol and embedded in paraffin to prepare a block. Subsequently, a 4 μm-thick tissue specimen was prepared using microtome, and then H&E (Hematoxylin & Eosin) staining, M-T (Masson-Trichrome) staining as collagen deposition staining, and PAS staining for observing goblet cells and secreted mucus were carried out. After the staining, observation was made using an optical microscope (Nikon, Japan) with a magnification of 400×.

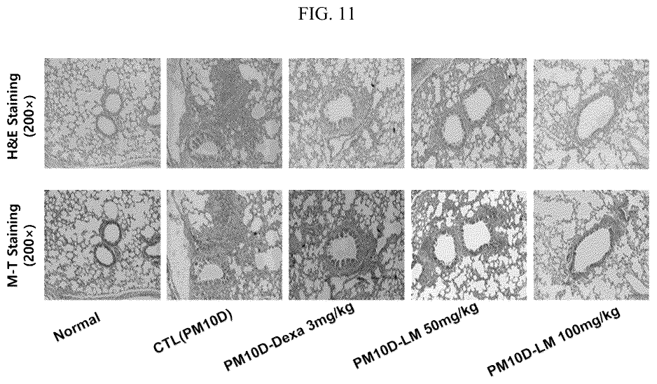

As a result, from the control group with induction by particulate matter, it was able to observe inflammatory cell infiltration and thickening of lung cell through the H&E staining, and also an increase in collagen fibers through the M-T staining. In contrast, the group administered with dexamethasone as a positive control group and the group administered with the Lysimachia mauritiana (LM) extract showed thinner wall of lung cells and also a decrease in the number of inflammatory cells and collagen fibers (FIG. 11).

### [Statistical Processing]

Results were recorded as mean±error, and significance was determined using Duncan's multiple comparison analysis method based on Student's T-test and one-way ANOVA (SPSS statistics version 19.0 statistic software, Inc, IBM, USA).

1-9. (canceled)

10: A method for treating or ameliorating a respiratory disease, the method comprising administering a Lysimachia mauritiana extract as an effective component to a subject in need thereof.

11: The method of claim 10, wherein the Lysimachia mauritiana extract is prepared by using water, C₁-C₄ lower alcohol, or a mixture thereof as an extraction solvent.

12: The method of claim 10, wherein the respiratory disease is selected from the group consisting of asthma, chronic obstructive pulmonary disease, bronchitis, pharyngitis, tonsillitis, laryngitis, and a combination thereof.

13: The method of claim 10, wherein the Lysimachia mauritiana extract is prepared by extracting an aerial part of Lysimachia mauritiana.

14: The method of claim 10, wherein the composition is prepared in a formulation selected from the group consisting of powder, granule, pill, tablet, capsule, candy, syrup, and beverage.

15: The method of claim 10, wherein the composition is included in a functional health food.

16: The method of claim 10, wherein the composition is a pharmaceutical composition, and further comprises a pharmaceutically acceptable carrier, excipient, and/or diluent.

17: The method of claim 10, wherein the subject is an animal.

18: The method of claim 10, wherein the composition is included in an animal feed.
